(19) United States
(12) Patent Application Publication
(10) Pub. No.: US 2005/0234440 A1
(43) Pub. Date: Oct. 20, 2005

(54) System with a sensor for perfusion management

(75) Inventor: Lowell L. Wood JR., Livermore, CA (US)

Correspondence Address: INTELLECTUAL VENTURES, LLC, 1756 - 114TH AVE., S.E., SUITE 110, BELLEVUE, WA, 98004, US

(73) Assignee: Searete LLC, a limited liability corporation of the State of Delaware

(21) Appl. No.: 10/827,578

(22) Filed: Apr. 19, 2004

(51) Int. Cl. A61B 18/04; A61B 5/00; A61M 31/00

(52) U.S. Cl. 606/34; 604/503; 606/27; 606/33

(57) ABSTRACT

A system for perfusion management that monitors, maintains, diagnoses, or treats perfusion deficiencies.

### CROSS-REFERENCE TO RELATED APPLICATIONS

The present application is related to, claims the earliest available effective filing date(s) from (e.g., claims earliest available priority dates for other than provisional patent applications; claims benefits under 35 USC § 119(e) for provisional patent applications), and incorporates by reference in its entirety all subject matter of the following listed applications; the present application also claims the earliest available effective filing date(s) from, and also incorporates by reference in its entirety all subject matter of any and all parent, grandparent, great-grandparent, etc. applications of the following listed applications:

1. U.S. patent application Ser. No. ______ entitled A SYSTEM FOR PERFUSION MANAGEMENT, naming Lowell L. Wood, Jr. as inventor, filed substantially contemporaneously and commonly assigned herewith.

2. U.S. patent application Ser. No. ______ entitled A SYSTEM WITH A RESERVOIR FOR PERFUSION MANAGEMENT, naming Lowell L. Wood, Jr. as inventor, filed substantially contemporaneously and commonly assigned herewith.

3. U.S. patent application Ser. No. ______ entitled A TELESCOPING PERFUSION MANAGEMENT SYSTEM, naming Lowell L. Wood, Jr. as inventor, filed substantially contemporaneously and commonly assigned herewith.

### TECHNICAL FIELD

The present application relates, in general, to detection and/or treatment.

### SUMMARY

In one aspect, a system includes but is not limited to: a body portion; at least one extensible finger coupled to said body portion; control circuitry coupled to said extensible finger or said body portion; and at least one sensor coupled to said extensible finger. In addition to the foregoing, other system aspects are described in the claims, drawings, and text forming a part of the present application.

In one aspect, a method includes but is not limited to: forming an elongating member for placement in proximity to a target location within an animal; coupling a signal detector for tracking a signal from said target location within said animal; and providing a positioning system communicating with said elongating member or said signal detector, said positioning system including logic or software configured for moving said elongating member to a second location cooperative with said signal from said target location. In addition to the foregoing, other method aspects are described in the claims, drawings, and text forming a part of the present application.

In another aspect, a method includes but is not limited to: generating a signal from a first location within an animal said signal retrievable by a signal capture tool coupled to a flexible conduit; transmitting said signal to a guiding system coupled to said flexible conduit; monitoring said signal at said second location or a new location; and performing an action with said guiding system responsive to said signal. In addition to the foregoing, other method aspects are described in the claims, drawings, and text forming a part of the present application.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In addition to the foregoing, various other method and or system aspects are set forth and described in the text (e.g., claims and/or detailed description) and/or drawings of the present application.

The foregoing is a summary and thus contains, by necessity; simplifications, generalizations and omissions of detail; consequently, those skilled in the art will appreciate that the summary is illustrative only and is NOT intended to be in any way limiting. Other aspects, inventive features, and advantages of the devices and/or processes described herein, as defined solely by the claims, will become apparent in the non-limiting detailed description set forth herein

### BRIEF DESCRIPTION OF THE FIGURES

FIG. 1 is a front-plan view of a device for perfusion management 100.

FIG. 2 is a front-plan view of another aspect of the device for perfusion management 100.

FIG. 3 is an exploded view of an extensible finger 104.

FIG. 4 is a schematic view of the control circuit 110 and devices in communication with the control circuit 110.

FIG. 5 illustrates an example wherein the device for perfusion management 100 is placed in a selected location in a human body 501.

The use of the same symbols in different drawings typically indicates similar or identical items.

### DETAILED DESCRIPTION

The present application uses formal outline headings for clarity of presentation. However, it is to be understood that the outline headings are for presentation purposes, and that different types of subject matter may be discussed throughout the application (e.g., device(s)/structure(s) may be described under the process(es)/operations heading(s) and/or process(es)/operations may be discussed under structure(s)/process(es) headings). Hence, the use of the formal outline headings is not intended to be in any way limiting.

1. Perfusion Management Device(s) and/or Process(es).

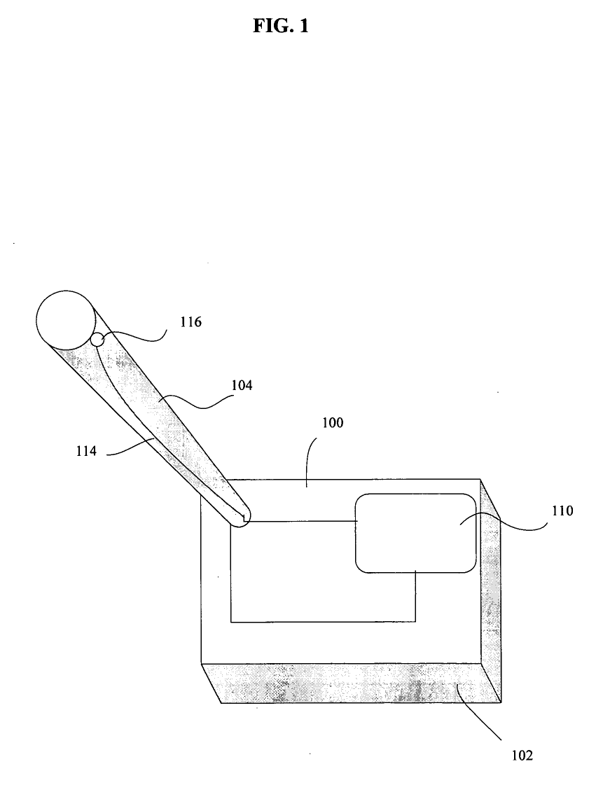

With reference now to FIG. 1, shown is a front plan view illustrative of various exemplary perfusion management device(s) and/or process(es). Accordingly, the present application first describes certain specific exemplary structures of FIG. 1; thereafter, the present application illustrates certain specific exemplary processes. Those having skill in the art will appreciate that the specific devices and processes described herein are intended as merely illustrative of their more general counterparts.

A. Structure(s) and or Device(s)

With reference to the figures, and with reference now to FIG. 1, shown is a front-plan view of a device for perfusion management 100. The device for perfusion management 100 includes a body portion 102 from which at least one extensible finger 104 projects. A sense line 114 connects a sensor 116 at the distal end of the extensible finger 104 to a control circuit 110.

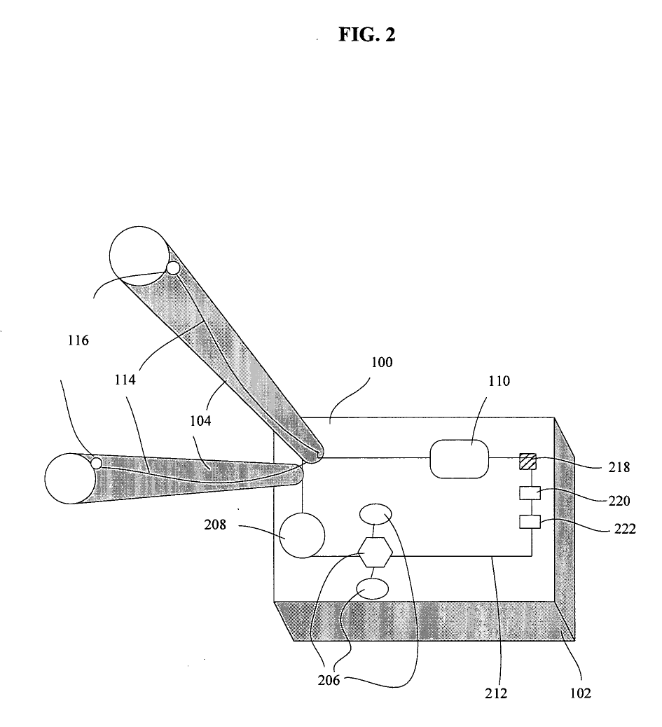

Referring now to FIG. 2, depicted is an aspect of the device for perfusion management 100 which includes the body portion 102 from which a set of at least one extensible finger 104 projects. In one aspect, each one of the extensible finger 104 of the set of at least one extensible finger has the sensor 116 at the distal end of the extensible finger 104. Additionally, a sense line 114 connects the control circuit 110 to the sensor 116.

Continuing to refer to FIG. 2, a receptacle 206 within the body portion 102 contains a fluid, for example, a fluid for treatment. A controllable valve 208 provides a path through which the fluid may travel to the at least one extensible finger 104. A control circuit 110 provides a control signal that may open or close the control valve 208.

Continuing to refer to FIG. 2, in one aspect, the at least one receptacle 206 may be coupled to a mixing chamber where the fluid contents of the at least one receptacle 206 are present for mixing and the mixed contents enter the extensible finger 104 for delivery to a selected location. In another approach, each of the extensible finger 104 of the multiple extensible finger 104 is in fluid communication with at least one of a respective receptacle 206 filled with a different fluid for delivery. The choice of the fluid in the at least one receptacle 206 may depend, for example, on the purpose of the device, for example, treatment of colon cancer, treatment of breast cancer, or treatment of arterial disease. The choice of fluid in the receptacle 206 includes, but is not limited to, for example, a chemical, a chemical compound, a protein, a lipoprotein, a glycoprotein, a sugar, a lipid, an antigen, an antibody, a cytokine, a peptide, a neurotransmitter, a hormone, an ion, a messenger a molecule, a nucleic acid, an engineered nucleic acid, a nucleic acid vector, a drug, a cell, a cell fragment, a cell organelle, a liposome, a pharmaceutical agent, a biological material, or a biological fraction. The receptacle 206 may also be utilized for storage and disposal of operational fluids. Also, although the exemplary embodiment described herein focuses primarily on fluid delivery, one skilled in the art will understand that fluid-like substances, such as gels, and fluidizable substances or non-fluid type substances, such as small solid particles, may be delivered in accordance with the invention. It will also be appreciated by those having skill in the art that the nature of the fluid in the receptacle 206 includes, for example, and is not limited to, a liquid, a solution, a mixture, a gel, a colloid, a colloid of a suitable viscosity, a suspension, an emulsion, or any material of low shear-strength for delivery to a site.

In one aspect one or more fluids are delivered to one or more of selected locations by the device for perfusion management 100. The selected location may be, for example, in proximity to or within a tumor, a circulatory system, an aorta, a vena cava, a site of therapy, or a site of investigation in an animal.

Continuing to refer to FIG. 2, a pump 218 provides fluid at a controlled flow rate for delivery to a site from the receptacle 206. It will be appreciated by those skilled in the art that the type of pump is not critical to the invention and may include, for example, a mechanical pump, a piezoelectric pump, an osmotic pump, a source of pressure, or a device for maintaining a positive flow of fluid through the device. Additionally, fluid flow may be further modulated with micro valves and self-pressurizing fluidic reservoirs. Moreover, in some applications, the fluid may be delivered without a pump. For example, fluid delivery may be controlled using a pressurized bladder, controlled dissolution or dilution of a material, a drip or gravity type of approach, or any other suitable approach to deliver an appropriate amount or an appropriate delivery-rate of the fluid.

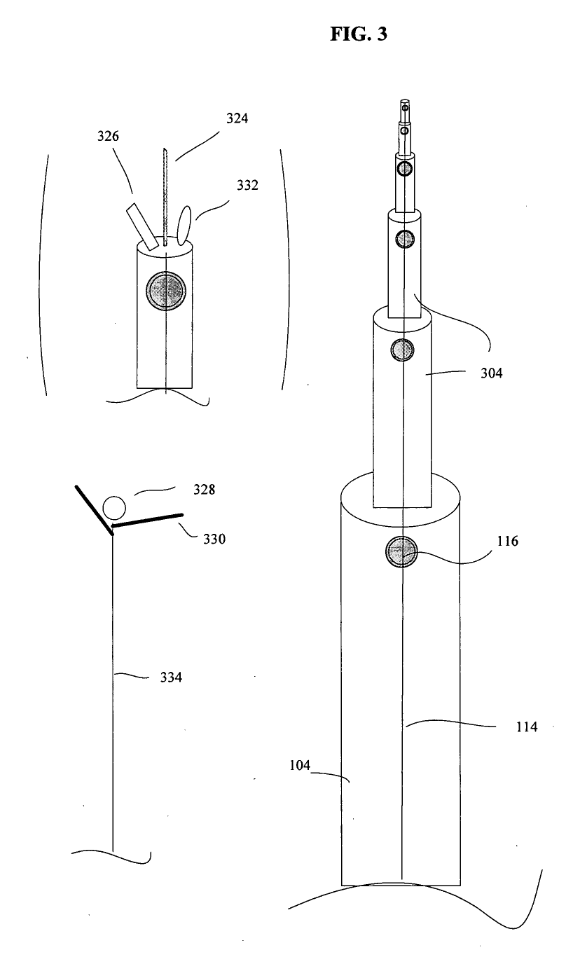

With reference now to FIG. 3, depicted is an exploded view of the extensible finger 104 showing a plurality of extended parts 304 with the sensor 116 at the distal end of each of the extended parts. In one aspect of the invention, the sensor 116 is an array of sensors, deployed from one or more portholes, at the distal end of each of the extended parts 304. In one approach, the portholes are sized and shaped to provide access through which the sensors 116 may be deployed. The portholes may include seals, stress relief or other features appropriate for proper mechanical deployment. In one approach, one or more of the portholes can be controllably opened or closed to provide communication exterior to the extensible finger or main body. The sensor 116 may be retracted within the porthole and deployed through the porthole. Where the porthole can be opened and closed, the porthole can close to limit communication and can be opened for deployment. The array of sensors may include, but is not limited to, for example, sensors for detecting pressure, temperature, chemical, gas, electrolyte, flow, volume, composition, or concentration. In an alternate aspect of the invention, microelectrodes, such as, for example, solid-state microelectrodes are sensitized with an agent for detecting a relevant interactor. Examples of the agent include, but are not limited to, for example, agonists of angiogenesis. The choice of sensor 116 depends on the physiological variable being monitored, treated, or controlled. The term “physiological variable” refers to any and all measurements relating to the functioning of a living organism in normal, sub-normal, or abnormal states.

Continuing to refer to FIG. 3, an operative tool 324 is coupled to the distal most extended part 304, or deployed from the porthole, or carried by the extensible finger 104, further including a carrying line 334 in communication with the control circuit 110. The operative tool 324 includes, but is not limited to, for example, one or more of a combination of, a tool positioner, an ablation device, a laser, a vacuum, a siphon 326, an evacuation device, a fluid dispenser 328, a cauterizer 330, a stent 332, a tissue-liquefying device, or a source of an electric or an electromagnetic charge 422. The vacuum, the siphon 326, or the evacuation device is employed for removing a cell, a mass of cells, a tissue, a fluid, a gel, a sample, a debris, a contaminant, or other material for which removal is desired or appropriate. The ablation device operates for perturbing or reducing the structural integrity or viability of a cell, a mass of cells, an assembly of biological materials exhibiting shear strength, or a tissue. The assembly of biological materials includes, for example, blood clots, cartilage, or bone. The source of an electric or electromagnetic charge 422 includes, but is not limited to, for example, steady state electric currents, time-varying electric currents, alternating electric currents, radio waves, microwaves, ultraviolet rays, infra-red rays, optical rays, terahertz beams, and the like.

Continuing to refer to FIG. 3, it will be appreciated by those having skill in the art that the operative tool 324 may include a set of devices having general or “multi-purpose” utility. The operative tool 324 may include, but is not limited to, for example, a combination of the fluid dispenser 328, the siphon 326, and the ablation device. In this example the operative tool combination, for example, delivers the fluid or gel, ablates cells, and removes debris.

Continuing to refer to FIG. 3, the plurality of extended parts 304 may themselves be hollow forming a conduit for delivery of the fluid to a site, or for housing a circuitry coupling the control circuit 110 to the operative tool 324, or for housing a mechanism that guides the extensible finger 104 or the plurality of extended parts 304.

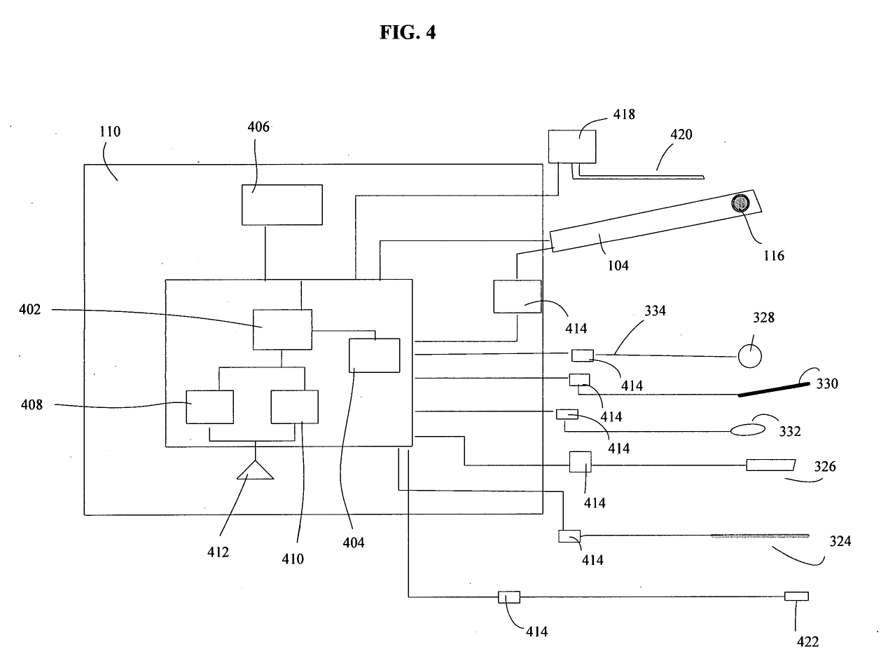

With reference now to FIG. 4, illustrated is a schematic view of the control circuit 110 and devices in communication with the control circuit 110. The device for perfusion management 100 shows a data transmitter 410, and a data receiver 408 coupled to the control circuit 110. An antenna 412 may be used for transmitting data to the exterior wirelessly. The antenna 412 is shown diagrammatically, but may be a structure, such as a strip antenna, that may be integrated in a manner that does not impair or significantly perturb system performance. The control circuit 110 is depicted as having a processor 402 coupled to a memory 404 that provides data storage and retrieval capability, and a power source 406. Feedback circuitry or logic circuitry provides communication between the control circuit 110 and devices in communication with it. In some applications, a software program providing instructions may be stored in the memory 404 to control operation of the control circuitry or to store data gathered under control of the control circuitry. Additionally, the control circuit 110 may have components for system integrated digital data gathering, processing, storage, compression and transmission. These can provide data control capabilities and operation control capabilities. For example, the transmission components may communicate through the antenna 412 to a person, system, computer, or device exterior to the body. This communication can allow data gathered by the sensors to be displayed, stored or otherwise processed in the external environment. Additionally, this communication may allow for the processed data or a plurality of new data to be received from the exterior by the device for perfusion management 100. Data compression can allow the control circuitry to store data representing larger amounts of data to be stored in the memory 404 or to be transmitted to the exterior environment in a more efficient manner.

Continuing to refer to FIG. 4, one or more of the operative tools 324 are mounted on an actuator 414 which allows for the independent movement of each tool. Alternatively, one or more operative tools 324 may be mounted as a unit on one actuator 414 and moved as a group, for example, forming an aspirating-dispensing unit. For example, the fluid dispenser 328 and the siphon 326 may be mounted together as a group. The actuator 414 may be a motor, a piezo electrically driven actuator, a micromechanical or electrical effector, or the like.

Continuing to refer to FIG. 4, the extensible finger 104 may include an imaging device deployed from the porthole or from the distal end of the extensible finger 104 or carried by a carrying line 334. The term “imaging device” being used herein to designate in general those components, circuits, assemblies and sub-assemblies comprising electrical, optical, acoustic, or opto-electronic components. In one aspect, the control circuit 110 is coupled to the imaging device that includes a laser 418, or a source of light or scene-illuminating radiation, coupled to an optical feed line 420 to illuminate an area. A charge coupled device is positioned to capture data from the illuminated area and provides an electronic signal indicative of the area imaged. Conventional circuitry then produces a digital representation that may be displayed, stored in the memory 404, or otherwise processed. The displayed image may serve, for example, for guiding the extensible finger 104 to the selected location or for determining the efficacy of a treatment or a procedure. One skilled in the art will recognize that the imaging device described herein is exemplary of imaging devices and that other imaging devices, including for example, raster and line-scanning imagers, nonvisible spectral imagers, and fluorescence imagers, may be included.

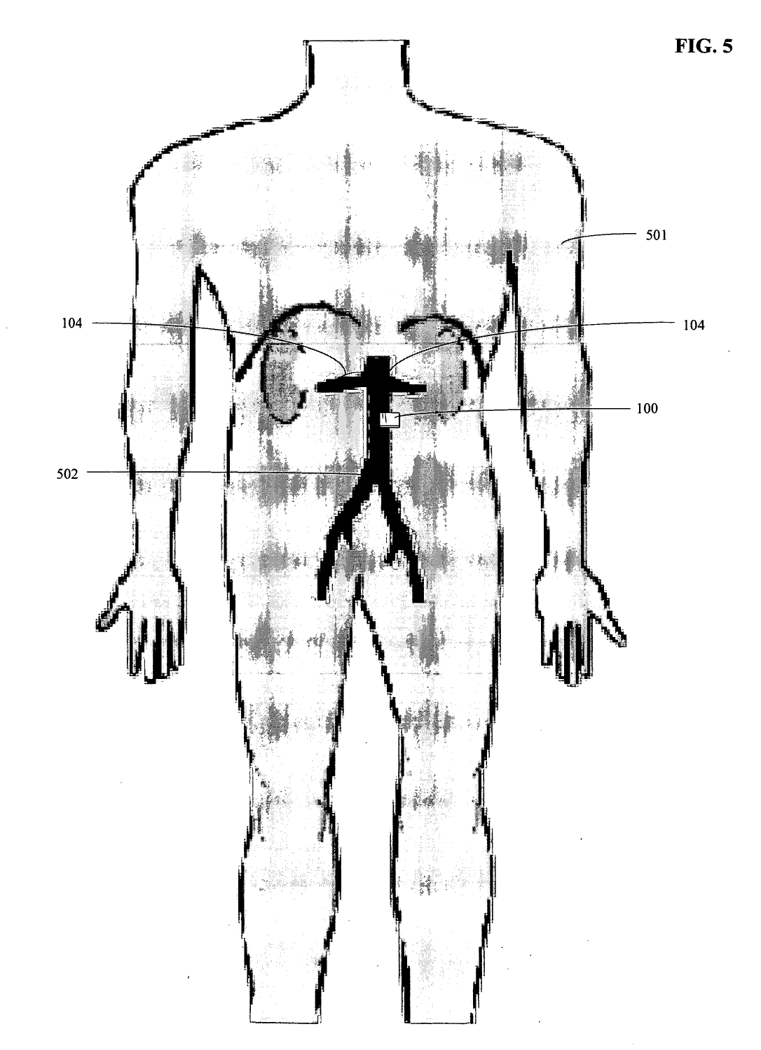

With reference now to FIG. 5, the device for perfusion management 100 is depicted implanted in an aorta 502 with the extensible finger 104 traveling a blood vessel in a human body 501. Additionally, the device for perfusion management 100 is configured for full or partial placement in the human body 501. The configuration may incorporate a combination of the following criteria, including but not limited to, dimensions, composition, shape, power dissipation level, or texture. In one aspect, the body portion 102 is sized for implantation in proximity to the aorta 502 or the vena cava and the extensible finger 104 is sized for traveling a blood vessel in an animal, for example, the human body 501. In this aspect, if the vasculature decreases two-fold, each of the extended parts 304 has about a two-fold decrease in diameter. The length of the extensible finger 104, for example, depends upon the distance between the selected location and the location of the body portion 102, and the route traveled by the extensible finger 104 to arrive at the selected location. It will be appreciated by those having skill in the art that the extensible finger 104 including the one or more of the operative tools 324 is of a size, dimension or shape operable for traveling one or more blood vessel of decreasing or increasing luminal diameter. It will also be appreciated by those having skill in the art that the extensible finger 104 and the one or more operative tool 324 may pass through the wall of the lumen, or trans-luminally, to the surrounding tissue for detecting, delivery of a treatment, or for sampling. It will also be appreciated by those having skill in the art that the trans-luminal mode described is not limited to blood vessels and includes the space or cavity of an organ or structure.

It will also be appreciated by those having skill in the art that the device for perfusion management 100 and its components, such as, for example, the extensible finger 104, the plurality of extended parts 304, or one or more operative tools 324, has a size, dimension, shape, material, and properties of flexion, retraction, and extension to allow for the steering, guiding, or positioning of the components of the device for perfusion management 100. For example, the extensible finger 104 may need to be steered around an occlusion or a fork in the vasculature. In this example, the extensible finger 104 may need to retracted, repositioned and then extended in a new direction. Extending, retracting or repositioning of the extensible finger 104 may be accomplished by techniques known in the art, for example, by using a guide wire or a by employing a shape polymer. In another aspect, the extensible finger may be retracted and then “punched through” an occlusion to dislodge it. In this example, lasers, shears, or a drug may be employed to degrade the occlusion. In this example, subsequent to the dislodgement and degradation of the occlusion, the siphon 326 or an evacuation device is employed to evacuate any debris, before the extensible finger 104 continues traveling the circulatory system. It will also be appreciated by those skilled in the art that the device for perfusion management 100 is not restricted to traveling the circulatory system but may be implanted in any tissue, such as, for example, nerve, epithelial, dermal, sub-dermal, connective, or muscle tissue. Additionally, the device for perfusion management 100 may be implanted in inter-tissue spaces, or inter-organ spaces, for example, those found within a body cavity.

In one aspect the device for perfusion management 100 includes an array of sensors 116 positioned across the plurality of extended parts 304 for monitoring, tracking, or mapping a gradient of temperature, pressure, or flow concentration in one or more locations. The one or more location may be, for example, a tissue, an artery, or a vein. In another aspect the device for perfusion management 100 has an auto-correct feature for correcting a sub-normal or abnormal gradient of temperature, pressure, flow or material concentration. In yet another aspect the device for perfusion management 100 has an auto-correct feature for detecting and correcting a sub-normal or abnormal gradient of temperature, pressure, flow, or material concentration.

The device for perfusion management 100 may be composed of materials known in the art, for example, a metal, a ceramic, a glass, a plastic, a polymer, a biologically compatible material, or a combination. For example, the device for perfusion management 100 may be made of helically-coiled stainless steel wire and coated with a polymer, such as, Teflon™. In another example, the device for perfusion management 100 may be made of helically-coiled stainless steel wire and coated with a polymer and impregnated with one or more of a biological material, for example, including but not limited to, anti coagulants, or inhibitors.

B. Operation(s) and/or Process(es)

Those having skill in the art will appreciate that some or all of the components of the device for perfusion management 100 may be present ex vivo. In one implementation, the device for perfusion management 100 is placed in proximity to the location on the animal, for example, the human body 501, and the extensible finger 104 directed to the selected location for detecting a level of pressure, temperature, chemical, gas, electrolyte, flow, volume, composition, or concentration. The extensible finger 104 may be retracted after such an operation, leaving the device for perfusion management 100 in place at the location, until time for a future operation or a new operation. The operation or the new operation includes but is not limited to, for example, repositioning of the extensible finger, or delivery of one or more of an effective agent in proximity to the location on the animal. In this implementation, the majority of the device for perfusion management 100 is ex vivo while the extensible finger 104 alternates between ex vivo and in vivo states.

In another aspect, some or all the components of the device for perfusion management 100 are present in vivo. In one implementation, the device for perfusion management 100 is placed in proximity to the location on the animal, for example, the human body 501, and the extensible finger 104 directed to the selected location for detecting a level of pressure, temperature, chemical, gas, electrolyte, flow, volume, composition, or concentration. The extensible finger 104 may be retracted after such an operation, leaving the device for perfusion management 100 in place at the location, until time for a future operation or a new operation. The operation or the new operation includes, but is not limited to, for example, repositioning of the extensible finger, or delivery of one or more of an effective agent in proximity to the location on the animal. The extensible finger 104 may be retracted after such a delivery, leaving the device for perfusion management 100 in place at the location, until time for a future delivery of the effective agent. In this implementation, the majority of the device for perfusion management 100 is in vivo while the extensible finger 104 alternates between retracted, partially retracted or unretracted states.

In one implementation, the device for perfusion management 100 is operable by a person. The person monitors, guides, positions, and performs other actions/operations or manages a response consistent with the device for perfusion management 100 being managed by the person. In such an implementation a separate display device can present imagery to aid the person. The imagery may be captured as described above with reference to FIG. 4, may be computer generated or may be captured by a separate imaging device internal to or external to the human body. Actions may be performed under control of the person who may be on site or may be linked from a remote location, or the device for perfusion management 100 may be programmed to perform some or all functions automatically. For example, the device for perfusion management 100 may be programmed to perform functions, such as, lumen clearance, lumen maintenance, monitoring of concentrations, sending of alerts, delivery of one or more of the effective agent at timed intervals or locations, self-check, and self-diagnosis. It will be appreciated by those of skill in the art that the device for perfusion management 100 may be programmed for complete auto operation of one or more functions.

C. Variation(s), and/or Implementation(s)

Those having skill in the art will recognize that the present application teaches modifications of the devices, structures, and/or processes within the spirit of the teaching herein. For example, the device for perfusion management 100 need not be limited to managing perfusion. The device provides a mechanism for exploring one or more regions and/or reaching a location within an animal, obtaining information, communicating this information, performing operations, performing procedures, and providing treatment. The treatment includes but is not limited to, for example, treatment of a subnormal, abnormal or pathological condition. In another example, the device for perfusion management 100 may find utility in the management of physiological functions, the detection or elimination of pathological functions or conditions, and/or treatment of a disease or a pathological state of non-human animals. Other modifications of the subject matter herein will be appreciated by one of skill in the art in light of the teachings herein.

The foregoing described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular aspects of the present subject matter described herein have been shown and described, it will be obvious to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is defined solely by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations), etc.

1. A device for perfusion management, comprising:
a body portion;
at least one extensible finger coupled to said body portion;
control circuitry coupled to said extensible finger or said body portion; and
at least one sensor coupled to said extensible finger.

2. The device according to claim 1, further comprising a pump, or a source of pressure coupled to said body portion or said at least one extensible finger.

3. The device according to claim 1, further comprising a motor, or an actuator coupled to said at least one extensible finger.

4. The device according to claim 1, further comprising an operative tool carried by at least one extensible finger.

5. The device according to claim 4, further comprising a tool positioner carried by at least one extensible finger.

6. The device according to claim 4, wherein said operative tool comprises a device for ablating, degrading, or liquefying a cell, a mass of cells, a tissue, or an assembly of biological materials exhibiting shear strength.

7. The device according to claim 4, wherein said control circuitry is operative to guide said operative tool.

8. The device according to claim 1, further comprising a source of an electric charge or an electromagnetic radiation coupled to said extensible finger in proximity to a location.

9. The device according to claim 1, further comprising a device for removing a cell, a tissue, a fluid, a gel, a colloid, an emulsion, a sample, a contaminant, a debris, or a biological material coupled to said at least one extensible finger.

10. The device according to claim 1, wherein said at least one extensible finger includes a plurality of extending parts.

11. The device according to claim 10, wherein said at least one plurality of extending parts is hollow.

12. The device according to claim 1, wherein said extensible finger is coupled to a device for fully or partially blocking or shunting a liquid flow.

13. The device according to claim 1, comprising a siphon, a vacuum, or an evacuation device coupled to said extensible finger.

14. The device according to claim 1, comprising a source of an electric charge or electromagnetic radiation coupled to said extensible finger.

15. The device according to claim 1, comprising a device to cauterize or seal a cell, a mass of cells, a tissue, or an assembly of biological materials exhibiting shear strength coupled to said extensible finger.

16. The device according to claim 1, comprising a stent coupled to said extensible finger.

17. The device according to claim 1, wherein said extensible finger is coated with a polymer or a biocompatible material.

18. The device according to claim 1, further comprising a fluid dispenser operative to provide a fluid at a controlled rate.

19. The device according to claim 18, wherein said fluid dispenser is carried by said at least one extensible finger.

20. The device according to claim 1, comprising a receptacle operative for storing a receivable.

21. The device according to claim 20, wherein said receptacle is carried by or coupled to said extensible finger.

22. The device according to claim 20 or 21, wherein said receptacle is coupled to said control circuitry.

23. The device according to claim 1, wherein said at least one extensible finger is coupled to a source of a chemical, a chemical compound, a protein, a lipoprotein, a glycoprotein, a sugar, a lipid, an antigen, an antibody, a cytokine, a peptide, a neurotransmitter, a hormone, an ion, a messenger molecule, a nucleic acid, an engineered nucleic acid, a nucleic acid vector, a drug, a cell, a cell fragment, a cell organelle, a liposome, a pharmaceutical agent, a biological material, or a biological fraction internal or external to said device.

24. The device according to claim 1, wherein said at least one extensible finger is coupled to a source of two or more of a chemical, a chemical compound, a protein, a lipoprotein, a glycoprotein, a sugar, a lipid, an antigen, an antibody, a cytokine, a peptide, a neurotransmitter, a hormone, an ion, a messenger molecule, a nucleic acid, an engineered nucleic acid, a nucleic acid vector, a drug, a cell, a cell fragment, a cell organelle, a liposome, a pharmaceutical agent, a biological material, or a biological fraction internal or external to said device.

25. The device according to claim 1, further comprising a data transmitter, coupled to said sensor or said control circuit.

26. The device according to claim 1, further comprising a data receiver coupled to said sensor or said control circuit.

27. The device according to claim 25 or 26, wherein said device communicates exterior to said animal.

28. The device according to claim 25, wherein said device is configured for monitoring or controlling by a person external to said animal.

29. The device for perfusion management according to claim 1, wherein said device is operative to provide a treatment or to monitor a response in said animal.

30. The device for perfusion management according to claim 29, wherein said treatment comprises delivering a medicinal agent, a pharmaceutical agent, a therapeutic device or assembly to a location in said animal.

31. The device for perfusion management according to claim 1, wherein said control circuitry comprises a processor, a feedback circuit, or a logic circuit.

32. The device for perfusion management according to claim 1, wherein said control circuitry is a processor further comprising a stored software or firmware program cooperative with said processor.

33. The device according to claim 1, wherein said control circuitry guides or moves said at least one extensible finger.

34. The device according to claim 1, wherein said at least one sensor comprises an imager, a pressure sensor, a temperature sensor, a chemical sensor, a gas sensor, an electrolyte sensor, a composition sensor, a concentration sensor, or a flow sensor.

35. The device according to claim 1, wherein said device is of a size, composition, power dissipation level, or shape configured for full or partial placement in vivo.

36. The device according to claim 1, wherein said device is coupled wirelessly for monitoring or controlling.

37. The device according to claim 1, wherein said device configured for implantation in said animal is of a dimension, a composition, a power dissipation level, or a shape appropriate for implantation in a selected location.

38. The device for perfusion management according to claim 37, wherein said selected location is in a circulatory system, an aorta, or a vena cava.

39. A method for fabricating a perfusion management device, comprising:
forming an elongating member for placement in proximity to a target location within an animal;
coupling a signal detector for tracking a signal from said target location within said animal; and
providing a positioning system communicating with said elongating member or said signal detector, said positioning system including logic or software configured for moving said elongating member to a second location cooperative with said signal from said target location.

40. The method according to claim 39, further comprising the step of coating said elongating member with a polymer or a biocompatible material.

41. The method according to claim 39, further comprising coupling a cavity to said elongating member for storing a receivable.

42. The method according to claim 41, comprising the step of coupling said cavity to a mixing cavity.

43. The method according to claim 41 or 42, including coupling a source of a chemical, a chemical compound, a protein, a lipoprotein, a glycoprotein, a sugar, a lipid, an antigen, an antibody, a cytokine, a peptide, a neurotransmitter, a hormone, an ion, a messenger a molecule, a nucleic acid, an engineered nucleic acid, a nucleic acid vector, a drug, a cell, a cell fragment, a cell organelle, a liposome, a pharmaceutical agent, a biological material, or a biological fraction internal or external to said cavity.

44. The method according to claim 41 or 42, including coupling a source of two or more of a chemical, a chemical compound, a protein, a lipoprotein, a glycoprotein, a sugar, a lipid, an antigen, an antibody, a cytokine, a peptide, a neurotransmitter, a hormone, an ion, a messenger a molecule, a nucleic acid, an engineered nucleic acid, a nucleic acid vector, a drug, a cell, a cell fragment, a cell organelle, a liposome, a pharmaceutical agent, a biological material, or a biological fraction internal or external to said cavity.

45. The method according to claim 39, comprising the step of coupling a device for partially blocking or shunting a liquid flow to said device.

46. The method according to claim 39, wherein a fluid dispenser is carried by said elongating member, said fluid dispenser including a fluid controller operative to provide a fluid at a controlled rate.

47. The method according to claim 39, comprising the step of coupling a motor or an actuator to said elongating member.

48. The method according to claim 39, comprising the step of coupling a source of an electric charge or an electromagnetic radiation to said elongating member.

49. The method according to claim 39, comprising the step of configuring said device to be monitored or controlled by a person.

50. The method according to claim 39, comprising the step of configuring said device for implantation in said predetermined location in said animal.

51. The method according to claim 39, including coupling a device for data gathering, processing, storage or transmission to positioning system.

52. The method according to claim 39, wherein said positioning system communicates wirelessly.

53. The method according to claim 39, wherein said positioning system is operative for guiding or moving said elongating member.

54. The method according to claim 39, wherein said positioning system comprises a processor, a feedback circuit, or a logic circuit.

55. The method according to claim 39, wherein said positioning system is a processor further comprising a stored software program cooperative with said processor.

56. The method according to claim 39, wherein said elongating member is formed from a plurality of flexible parts.

57. The method according to claim 56, wherein said plurality of flexible parts are hollow.

58. The method according to claim 39, comprising the step of coupling an operative tool to said elongating member.

59. The method according to claim 58, wherein said operative tool comprises, a device for ablating, or a device for degrading a cell, a mass of cells, a tissue, or an assembly of cells exhibiting shear strength.

60. The method according to claim 39, comprising the step of coupling a vacuum, a siphon, or an evacuation device to said elongating member.

61. The method according to claim 39, comprising coupling a device for cauterizing or sealing a cell, a mass of cells, a tissue, or an assembly of cells exhibiting shear strength to said elongating member.

62. The method according to claim 39, comprising the step of coupling a stent to said elongating member.

63. The method according to claim 39, wherein said signal detector includes an imager, a pressure sensor, a temperature sensor, a chemical sensor, a gas sensor, an electrolyte sensor, a sensor of composition, a sensor of concentration, or a flow sensor coupled to said elongating member.

64. The method according to claim 39, further comprising the step of configuring said device for managing a response.

65. A method for managing perfusion, comprising:
generating a signal from a first location within an animal said signal retrievable by a signal capture tool coupled to a flexible conduit;
transmitting said signal to a guiding system coupled to said flexible conduit;
monitoring said signal at said second location or a new location; and
performing an action with said guiding system responsive to said signal.

66. The method for perfusion management according to claim 65, comprising the step of detecting a level of pressure, temperature, chemical, gas, composition, concentration, electrolyte, or flow.

67. The method for perfusion management according to claim 65, wherein said action further comprises the step of moving or positioning said flexible conduit.

68. The method for perfusion management according to claim 65, wherein said action further comprises the step of delivering a chemical, a chemical compound, a protein, a lipoprotein, a glycoprotein, a sugar, a lipid, an antigen, an antibody, a cytokine, a peptide, a neurotransmitter, a hormone, an ion, a messenger a molecule, a nucleic acid, an engineered nucleic acid, a nucleic acid vector, a drug, a cell, a cell fragment, a cell organelle, a liposome, a pharmaceutical agent, a biological material, or a biological fraction.

69. The method for perfusion management according to claim 65, wherein said action further comprises the step of delivering two or more of a chemical, a chemical compound, a protein, a lipoprotein, a glycoprotein, a sugar, a lipid, an antigen, an antibody, a cytokine, a peptide, a neurotransmitter, a hormone, an ion, a messenger a molecule, a nucleic acid, an engineered nucleic acid, a nucleic acid vector, a drug, a cell, a cell fragment, a cell organelle, a liposome, a pharmaceutical agent, a biological material, or a biological fraction.

70. The method for perfusion management according to claim 65, wherein said action further comprises the step of delivering an electric current or an electromagnetic radiation in proximity to a cell, a mass of cells, a tissue, or an assembly of biological materials exhibiting shear strength.

71. The method for perfusion management according to claim 65, wherein said action further comprises the step of directing, blocking, or shunting a liquid flow.

72. The method for perfusion management according to claim 65, wherein said action further comprises the step of ablating, degrading, or liquefying a cell, a mass of cells, a tissue, or an assembly of materials exhibiting shear strength.

73. The method for perfusion management according to claim 65, wherein said action further comprises the step of evacuating a target.

74. The method for perfusion management according to claim 65, wherein said action further comprises the step of cauterizing or sealing a cell, a mass of cells, a tissue, or an assembly of materials exhibiting shear strength.

75. The method for perfusion management according to claim 65, wherein said action further comprises the step of dispensing a fluid at a controlled rate.
